(19) United States
(12) Patent Application Publication
(10) Pub. No.: US 2026/0199659 A1
(43) Pub. Date: Jul. 16, 2026

(54) IMPELLER FOR BLOOD PUMPS, RELATED CAGES AND ASSEMBLIES

(71) Applicant: FBR Medical, Inc., El Dorado Hills, CA (US)

(72) Inventors: Marius KORFANTY, Dresden (DE); Ralph-Peter MUELLER, Brooklyn, NY (US)

(21) Appl. No.: 19/137,421

(22) PCT Filed: Dec. 5, 2023
(86) PCT No.: PCT/US2023/082427

§ 371 Date: Jun. 10, 2025

Related U.S. Application Data
(60) Provisional application No. 63/476,025, filed on Dec. 19, 2022.

(51) Int. Cl. A61M 60/806; A61M 60/13

(52) U.S. Cl. CPC A61M 60/806; A61M 60/13

(57) ABSTRACT

Impellers and cooperating impeller cages for blood pumps that include an impeller flow path contour with an exit impeller blade segment that extends radially outward from a nose and terminates at a straight outer edge that is parallel to an axial centerline of the impeller and faces windows in the impeller cage to provide improved outflow and/or better hydrodynamic efficiency from current commercial devices.

### RELATED APPLICATIONS

The present application claims the benefit of and priority to U.S. Provisional Patent Application Ser. No. 63/476,025, filed Dec. 19, 2022, the contents of which are hereby incorporated by reference as if recited in full herein.

### FIELD OF THE INVENTION

The present invention relates to blood pumps and is particularly suitable for intra-vascular blood pumps such as catheter blood pumps.

### BACKGROUND

Over the years, various types of blood pumps have been developed for the purpose of augmenting or replacing the blood pumping action of damaged or diseased hearts. The pumps may be designed to provide right and/or left ventricular assist, although left ventricle assist is the most common application in that it is far more common for the left ventricle to become diseased or damaged than it is for the right ventricle.

Blood pumps must pump the fluid at a suitable rate without applying excessive Reynolds shear stress to the fluid. It is well known to those skilled in the art that lysis or cell destruction may result from application of shear stress to cell membranes. Red blood cells are particularly susceptible to shear stress damage as their cell membranes do not include a reinforcing cytoskeleton to maintain cell shape.

Intravascular blood pumps comprise miniaturized blood pumps capable of being percutaneously or surgically introduced into the vascular system of a patient, typically to provide left and/or right heart support. See, e.g., U.S. Pat. No. 4,625,712 which describes a multiple stage intravascular axial-flow blood pump which can be percutaneously inserted into an artery for heart assist and U.S. Pat. No. 4,846,152 which describes a single-stage intravascular axial flow blood pump, the contents of which are hereby incorporated by reference as if recited in full herein. These blood pumps position the drive unit/motor outside the body (extracorporeal) and use long cable drive systems. The maneuverability and/or durability of these types of blood pumps was often less than desired. During use, components of these devices tended to deteriorate prematurely due to rotational and pulsatile forces experienced by the blood pumps.

Other intravascular blood pumps are configured so that the drive unit/motor and the impeller are directly connected to each other, with the motor and the impeller (pump) housing having the substantially the same outer diameter. See, e.g., U.S. Pat. No. 6,176,848, the contents of which are hereby incorporated by reference as if recited in full herein.

While these systems have been used successfully to pump blood, the flow rates provided are believed to typically be under 3 liters/minute (except for peak output at limited duration) at a counterpressure of about 100 mm Hg. The pumping rate is limited by the torque limitation of the small “micro” motors and/or the configuration of the impeller.

There is a need for blood pumps that can provide increased blood flow rates without applying undue shear forces.

### SUMMARY

Embodiments of the present invention provide impellers with curvilinear profiles with cooperating relatively large windows of an impeller cage for blood outflow of blood pumps.

The curvilinear profiles can include an exit blade segment with a straight outer edge that has a length that is about 50-110% of a length of a window of an impeller cage.

The impeller cage can have only three struts, with a window extending between neighboring struts. The impeller cage can also have only three circumferentially spaced apart windows.

The impeller can be in communication with a motor that rotates the impeller to pump blood into a heart of a subject.

The impeller can be in communication with an extracorporeal motor. A multi-lumen shaft can enclose a long torque cable that is coupled to the motor at one end and to an impeller shaft of the impeller at another end and that provides in-flow fluid path(s) and an out flow (purge) fluid path(s) that cool and/or lubricate the long torque cable.

The impeller can be in communication with an adjacent, in vivo motor that does not require a long drive cable.

Embodiments of the present invention are directed to an impeller for a blood pump that includes: an impeller body with a curvilinear profile that extends from a distal nose segment to a pair of vanes positioned proximally of the nose segment. Each vane provides an exit blade segment that has a constant, maximum radial extent measured from an axially extending centerline of the impeller body over a length of the exit blade segment.

The length of the exit blade segment can be in a range of 3.7 mm to 3.8 mm.

The exit blade segment can be perpendicular to the axially extending centerline on all spans.

The impeller blade can have an axial length that is about 9.15 mm.

The impeller blade can have a wrap angle of about 130 degrees.

The impeller can be provided in combination with an impeller cage at least partially surrounding the impeller body. The exit blade segment can be longitudinally aligned with cage windows of the impeller cage.

The impeller cage can have only three windows that can be longitudinally aligned and circumferentially spaced apart and three struts that are longitudinally aligned and circumferentially spaced apart.

The three windows can each have a longitudinally extending length and a circumferentially extending width defining a respective window area. The window area can be in a range of 10 mm² to 14 mm².

The exit blade segments can have a length that is about 80-110% of a length of the windows.

The length of the exit blade segment can be the same as the length of the three windows.

The cage windows can have a circumferential peripheral angle that can be in a range of 90-100 degrees.

The circumferentially extending peripheral angle can be about 98 degrees.

The impeller body can have an overall length in a range of 8.5 mm to 9.5 mm.

The overall length can be about 9.15 mm.

The impeller can be configured to taper out from the nose segment to spaced apart, longitudinally aligned peak segments, then taper radially inward in a proximal direction to merge into the vanes. The vanes can have a radius equal to a maximal radius of the peak segment.

The impeller cage defines a stage length (Lstage) that is greater than an overall length of the impeller. The stage length can be greater in length that the impeller in an amount that is less than 0.25 mm.

The impeller can have an impeller cage surrounding the impeller. A clearance distance between an inner surface of the impeller cage and the peak segments can be about 0.75 mm.

The impeller body can have a maximal outer diameter of about 4.15 mm.

The impeller cage can have a window area/cylinder area ratio of 0.8043.

The exit blade segments can terminate adjacent the proximal end of the windows.

Yet other embodiments are directed to an impeller assembly for a catheter blood pump. The impeller assembly has a curvilinear profile that extends from a distal nose segment to a pair of vanes positioned proximally. Each vane provides an exit blade segment that has a constant, maximum radial extent measured from an axially extending centerline of the impeller body over a length of the exit blade segment. The impeller assembly also includes an impeller cage at least partially surrounding the impeller. The exit blade segment is longitudinally aligned with cage windows of the impeller cage.

The impeller cage can have only three windows that are longitudinally aligned and circumferentially spaced apart and three struts that are longitudinally aligned and circumferentially spaced apart.

The three windows each have a longitudinally extending length and a circumferentially extending width defining a respective window area. The window area of each window can be in a range of 10 mm² to 14 mm².

The exit blade segments can have a length that is about 80-110% of a length of the windows.

The exit blade segments can define an exit flow angle of 90 degrees.

The length of the exit blade segments can be the same as the length of the three windows.

The cage windows can have a circumferential peripheral angle that is in a range of 90-100 degrees.

The circumferential peripheral angle can be 98 degrees.

The impeller can have an overall length in a range of 8.5 mm to 9.5 mm.

The overall length can be about 9.15 mm.

The nose segment can merge into an adjacent blade segment that tapers out to a peak segment. The blade can then taper radially inward in a proximal direction, then merge into the vanes. The vanes can have a radius corresponding to (substantially the same as a maximal radius) of the peak segment.

The cage defines a stage length (Lstage) that can be greater than an overall length of the impeller in an amount that is less than 0.25 mm.

A clearance distance between an inner surface of the impeller cage and the peak segment(s) can be about 0.75 mm.

The impeller body can have a maximal outer diameter of about 4.15 mm.

The impeller cage can have a window area/cylinder area ratio of 0.8043.

The exit blade segments can terminate adjacent the proximal end of the windows.

The impeller cage can have only three windows that are longitudinally aligned and circumferentially spaced apart and only three struts that are longitudinally aligned and circumferentially spaced apart.

The windows can have top and bottom perimeters that are straight in a circumferential dimension.

The three windows can each have a longitudinally extending length and a circumferentially extending width defining a respective window area that can be in a range of 10 mm² to 14 mm².

The cage windows have a circumferential peripheral angle that can be in a range of 90-100 degrees.

The circumferential peripheral angle can be about 98 degrees.

The three struts can have a circumferentially extending width that is in a range of 0.4 mm to 0.7 mm and a longitudinally extending length that is in a range of 3.7 mm to 4.0 mm.

The length of the struts can be 3.75 mm.

Yet other embodiments are directed to an in vivo impeller for a blood pump having an impeller body configured with an exit blade having a straight exit blade angle of 90 degrees over at least 30% of a length of the impeller body.

Further features, advantages and details of the present invention will be appreciated by those of ordinary skill in the art from a reading of the figures and the detailed description of the preferred embodiments that follow, such description being merely illustrative of the present invention.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

### BRIEF DESCRIPTION OF DRAWINGS

FIG. 1 is a side view of a blood pump, with one part of a shell handle omitted to reveal internal components, according to embodiments of the present invention.

FIG. 2 is another side view of the blood pump shown in FIG. 1, rotated at 90 degrees from the orientation shown in FIG. 1.

FIGS. 3A-3C are enlarged views of a prior art axial impeller and cage of a catheter blood pump.

FIG. 4A is an enlarged side view of an impeller and cage according to embodiments of the present invention.

FIG. 4B is a side perspective view of the impeller and cage shown in FIG. 4A.

FIG. 4C is a distal end view of the impeller and cage shown in FIG. 4A.

FIG. 4D is a side perspective view of the impeller shown in FIG. 4A.

FIG. 4E is a side perspective view of the cage shown in FIG. 4A.

FIG. 4F is a schematic illustration of turbomachinery fundamentals of outlet angles corresponding to the impeller according to embodiments of the present invention.

FIG. 4G is a schematic illustration of a portion of the impeller providing blade segments define an exit flow angle of 90 degrees according to embodiments of the present invention.

FIG. 4H is a side perspective view of the impeller and impeller cage (transparent) showing virtual intersecting lines corresponding to the exit flow angle shown in FIG. 4G.

FIG. 4I is a distal end view of the impeller and impeller cage shown in FIG. 4H with virtual intersecting lines corresponding to the exit flow angle shown in FIG. 4G.

FIG. 5A is an enlarged side view of the impeller and cage shown in FIGS. 4A-4C but shown adjacent FIG. 6A for ease of reference.

FIG. 5B is an end view of the impeller and cage shown in FIG. 5A and shown adjacent FIG. 6B for ease of reference.

FIG. 6A is an enlarged side view of the prior art impeller and cage shown in FIGS. 3A-3C.

FIG. 6B is an end view of the prior art impeller and cage shown in FIG. 6A.

FIG. 7 is a table of example parameters of the impeller and cage shown in FIGS. 5A/5B (left side column) and FIGS. 6A/6B (right side column) according to embodiments of the present invention.

FIG. 8 is a graph of meridional contour of the impeller blade shown in FIG. 4D.

FIG. 9 is a graph of meridional contour of the prior art impeller shown in FIGS. 3A-3C.

FIG. 10 is a side perspective view of an impeller illustrating meridional flow surfaces with meridional and tangential coordinates to a local radius that can be used to map to a plane by a coordinate transformation.

FIG. 11 is a graph of blade mean lines (inner, mid, outer spans) m, t of the impeller shown in FIGS. 4A-4C and the prior art impeller shown in FIGS. 3A-3C.

FIG. 12 is a graph of axial pump impeller comparison of the impellers shown in FIGS. 3A-3C and 4A-4C, bland angle β-distribution (inner, mid, outer spans) (P vs m/Max), according to embodiments of the present invention.

FIGS. 13-16 are side perspective views of example impeller cage configurations according to embodiments of the present invention.

FIG. 17 is a side perspective view of a prior art impeller cage.

FIG. 18A is an enlarged side perspective view of finite element analysis (FEA) results of the narrow three strut impeller cage shown in FIG. 13, color-coded with vonMises stress results (psi) according to embodiments of the present invention.

FIG. 18B is a greatly enlarged proximal segment of the impeller cage FEA results shown in FIG. 18A.

FIG. 18C is an enlarged proximal portion of a strut shown in FIG. 18B.

FIG. 19A is a side perspective view of FEA results, color-coded with URES, mm deflection, of the narrow three strut impeller cage shown in FIG. 18A, illustrating a deflection graphically amplified by 50× according to embodiments of the present invention.

FIG. 19B is a side perspective view of FIG. 19A, modified to show “true deflection”, reducing clearance to the impeller.

FIG. 20A is an enlarged side perspective view of finite element analysis (FEA) results of the wider three strut impeller cage shown in FIG. 14, color-coded with vonMises stress results (psi) according to embodiments of the present invention.

FIG. 20B is a greatly enlarged proximal segment of the impeller cage FEA results shown in FIG. 20A.

FIG. 20C is an enlarged proximal portion of a strut shown in FIG. 20A.

FIG. 21A is a side perspective view of FEA results, color-coded with URES, mm deflection, of the wider three strut impeller cage shown in FIG. 14, illustrating a deflection graphically amplified by 70× according to embodiments of the present invention.

FIG. 21B is a side perspective view of the impeller cage and FEA results shown in FIG. 21A, modified to show “true deflection”, reducing clearance to the impeller.

FIG. 22A is an enlarged side perspective view of finite element analysis (FEA) results of the narrow four strut impeller cage shown in FIG. 15, color-coded with vonMises stress results (psi) according to embodiments of the present invention.

FIG. 22B is a greatly enlarged proximal segment of the impeller cage FEA results shown in FIG. 22A.

FIG. 22C is an enlarged proximal portion of a strut shown in FIG. 22B.

FIG. 23A is a side perspective view of FEA results, color-coded with URES, mm deflection, of the narrow four strut impeller cage shown in FIG. 15, illustrating a deflection graphically amplified by 70× according to embodiments of the present invention.

FIG. 23B is a side perspective view of FIG. 23A, modified to show “true deflection”, reducing clearance to the impeller.

FIG. 24A is an enlarged side perspective view of finite element analysis (FEA) results of the wider, four strut impeller cage shown in FIG. 16, color-coded with vonMises stress results (psi) according to embodiments of the present invention.

FIG. 24B is a greatly enlarged proximal segment of the impeller cage FEA results shown in FIG. 24A.

FIG. 25A is a side perspective view of FEA results, color-coded with URES, mm deflection, of the wider four strut impeller cage shown in FIG. 16, illustrating a deflection graphically amplified by 70× according to embodiments of the present invention.

FIG. 25B is a side perspective view of FIG. 25A, modified to show “true deflection”, reducing clearance to the impeller.

FIG. 26A is a greatly enlarged side perspective view of an example impeller housing/outlet cage according to embodiments of the present invention.

FIG. 26B is a greatly enlarged side view of the example impeller housing/outlet cage shown in FIG. 26A.

FIG. 27A is a greatly enlarged side perspective view of another example impeller housing/outlet cage according to embodiments of the present invention.

FIG. 27B is a greatly enlarged side view of the example impeller housing/outlet cage shown in FIG. 27A.

### DETAILED DESCRIPTION

The present invention will now be described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. The abbreviation “FIG.” may be used interchangeably with “Fig.” and the word “Figure” in the specification and figures. It will be appreciated that although discussed with respect to a certain embodiment, features or operation of one embodiment can apply to others.

In the drawings, the thickness of lines, layers, features, components and/or regions may be exaggerated for clarity and broken lines (such as those shown in circuit of flow diagrams) illustrate optional features or operations, unless specified otherwise. In addition, the sequence of operations (or steps) is not limited to the order presented in the claims unless specifically indicated otherwise.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when a feature, such as a layer, region or substrate, is referred to as being “on” another feature or element, it can be directly on the other element or intervening elements may also be present. In contrast, when an element is referred to as being “directly on” another feature or element, there are no intervening elements present. It will also be understood that, when a feature or element is referred to as being “connected” or “coupled” to another feature or element, it can be directly connected to the other element or intervening elements may be present. In contrast, when a feature or element is referred to as being “directly connected” or “directly coupled” to another element, there are no intervening elements present. Although described or shown with respect to one embodiment, the features so described or shown can apply to other embodiments. The term “about” means that the noted number can vary by +/−20%.

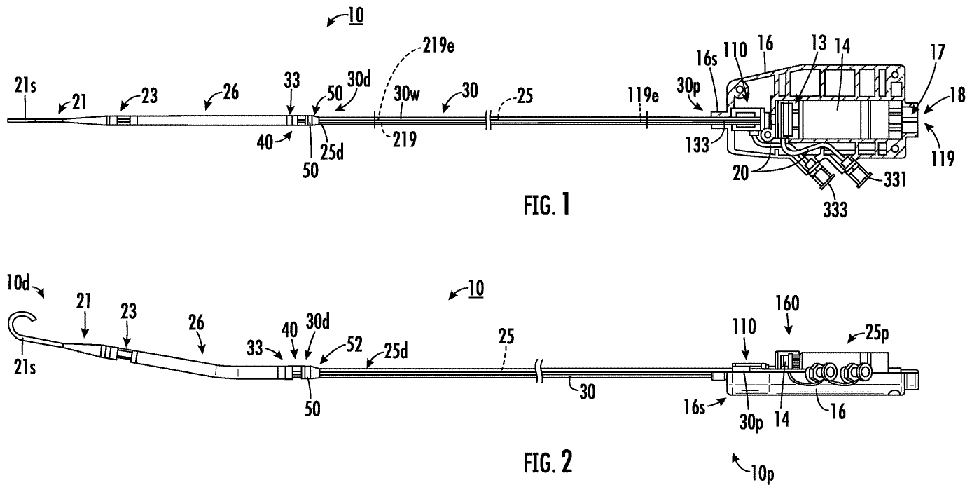

Referring to FIGS. 1 and 2 an example blood pump 10 is shown. The blood pump 10 comprises a motor 14, a multi-lumen shaft 30 that encloses a torque cable 25, an inlet cage 23 (blood intake), a (snorkel) tube 21 extending between the inlet cage 23 and an impeller 40, and an impeller housing/outlet cage 33 (pumped blood outlet). A snorkel 21s can be attached to the snorkel tube 21 and be positioned at a distal end 10d of the blood pump 10. The snorkel/snorkel tube may be provided in a number of configurations. The snorkel tube 21 may merge into or comprise a pigtail. The snorkel tube 21 may merge distally into or comprise a circular 3-D spacer. See, U.S. Provisional Application Ser. No. 63/518,163, filed Aug. 8, 2023, the contents of which are hereby incorporated by reference as if recited in full herein.

The blood pump 10 can also have a manifold 110 that is coupled to the motor 14. The manifold 110 has a manifold chamber. The manifold 110 can sealably enclose a sub-length of the multi-lumen shaft 30, typically at least a segment of the proximal end portion 30p of the multi-lumen shaft 30 and can define at least a portion of a (purge) fluid in-flow path of the multi-lumen shaft 30 that extends into at least one in-flow lumen(s) 133 provided by the multi-lumen shaft 30. The term “in-flow” can be used interchangeably with the term “inflow” herein. The term “out-flow” can be used interchangeably with the term “outflow” herein.

In example embodiments, the blood pump 10 can also have a bearing housing 50 adjacent the impeller 40 with a bearing housing adapter 52 that couples an outer wall 30w of the multi-lumen shaft 30 to the bearing housing 50.

The multi-lumen shaft 30 has a proximal end portion 30p that is adjacent the motor 14 and an opposing distal end portion 30d that terminates adjacent the impeller 40. The torque cable 25 also has a proximal end portion 25p that is adjacent the motor 14 and an opposing distal end portion 25d that terminates adjacent the impeller 40. The torque cable 25 can also be interchangeably referred to as a “drive cable”. The torque cable 25 can be directly or indirectly attached to the impeller 40 at the distal end portion 25d of the torque (drive) cable 25 and to the motor 14 at the proximal end portion 25p of the torque (drive) cable 25.

The motor 14 can be held in a housing 16. The housing 16 can be provided as a cooperating pair of handle shells 16s.

An intrabody portion of the blood pump 10 (distal to the housing 16) is configured to be inserted into the aorta from a remote entry point, such as an incision below the groin that provides access into a femoral artery. The intrabody portion of the blood pump 10 (snorkel 21 leading the way), then passes through the descending aorta until it reaches the ascending aorta, near the heart. The multi-lumen shaft 30 encloses the torque cable 25 and can have a length sufficient to position the motor 14 to be extracorporeal. The proximal end portion 30p of the multi-lumen shaft 30 can reside outside the body, typically near the patient's groin, at an end portion opposing the impeller 40 and snorkel 21.

In an example operational configuration, the blood intake cage 23 and snorkel 21 reside in a left ventricle (LV) of a heart of a patient, the impeller 40 and blood outlet cage 33, also interchangeably referred to herein as “impeller cage”, positioned in the aorta, proximate the coronary arteries, above the aortic valve to discharge pumped blood into the aorta. The motor 14 can reside inside the patient adjacent the impeller 40. However, it may be preferred to have an extracorporeal motor for improved torque output. Thus, in some embodiments, the motor 14 and motor housing 16 can be outside the patient.

Generally stated, when the proximal end portion of the torque cable 25 is mechanically rotated by a motor shaft of the motor 14, typically located outside the patient's body, it conveys the rotational force through the length of the multi-lumen shaft 30, causing the impeller 40 to spin at high speed in or near the heart.

The blood pump 10 can be particularly suitable in providing ventricular assist during surgery or providing temporary bridging support to help a patient survive a crisis.

The motor 14 is arranged to drive the torque cable 25 in the multi-lumen shaft 30 which in turn drives the impeller 40/pump unit. The motor 14, if operated at an extracorporeal site, can have any desired size. The multi-lumen shaft 30 provides continuous lubrication by a biocompatible (purge) liquid. A part of this liquid can exit through a bearing housing/impeller shaft interface and thus enter the blood stream. The remaining part can be directed to flow through an out-flow path and be collected extracorporeally after passing through a lumen provided in the multi-lumen shaft 30 that holds the drive cable 25.

The multi-lumen shaft 30 and the impeller 40 may be dimensioned to any suitable diameter for intravascular applications. For example, the range of sizes may include, but is not necessarily limited to, 9 French to 30 French, although the range is typically in a range of 12 French to 24 French, and more typically in a range of 12 French to 18 French. Cardiologists can thus insert the small CBP device minimally invasively. In some preferred embodiments, the diameter of can be 12 French (4.0 mm) or less providing a low-profile device that can minimize bleeding.

The blood pump 10 can comprise first and second support wires 119, 219 that are longitudinally spaced apart and reside inside the torque cable 25. Referring to FIG. 1, the first support wire 119 can have a distal end 119 end that terminates a range of 1-3 inches from the manifold 110 and extends at least partially through the motor shaft. The second support wire 219 can have a proximal end 219e that terminates a range of 1-3 inches from the proximal end of the impeller shaft. The first support wire 119 can support the torque cable 25 at a high torque area (at the motor 14) so that the torque cable 25 does not collapse under load. The first support wire 119 can also act as a strain relief when it exits a distal end of the manifold 110. The second support wire 219 can allow the impeller shaft and torque cable 25 to be crimped together by using a proximal bushing without collapsing the (hollow) torque cable 25. The second support wire 219 can also act as a strain relief.

In some embodiments, the first and second support wires 119, 219 can be provided as a single support wire instead of separate support wires and the single support wire may extend substantially an entire length of the torque cable 25 or reside only at a proximal end portion or only at a distal end portion of the torque cable 25.

Further details of an example blood pump 10 are provided in U.S. Provisional Patent Application Ser. No. 63/374,426 filed Sep. 2, 2022, and PCT/US23/21351, filed May 8, 2023, the contents of which are hereby incorporated by reference as if recited in full herein. However, the impeller 40 and outlet/impeller cage 33 can be used with other blood pump systems and are not limited to that disclosed in the noted provisional patent application.

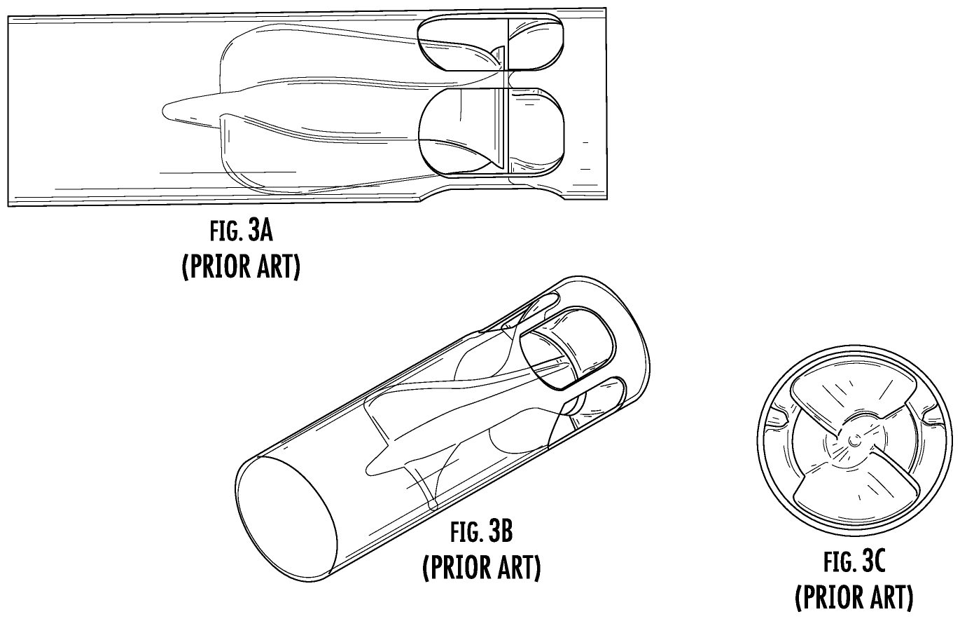

Turning now to FIGS. 3A-3C, a prior art impeller and cage of a blood pump are shown. The inventors have engaged in extensive research and development efforts to develop an impeller 40 with a novel impeller configuration as well as novel configurations of an impeller cage 33 which, based on computational fluid (hydro) dynamic evaluations, provide improved flow over the prior art impeller and cage shown in FIGS. 3A-3C.

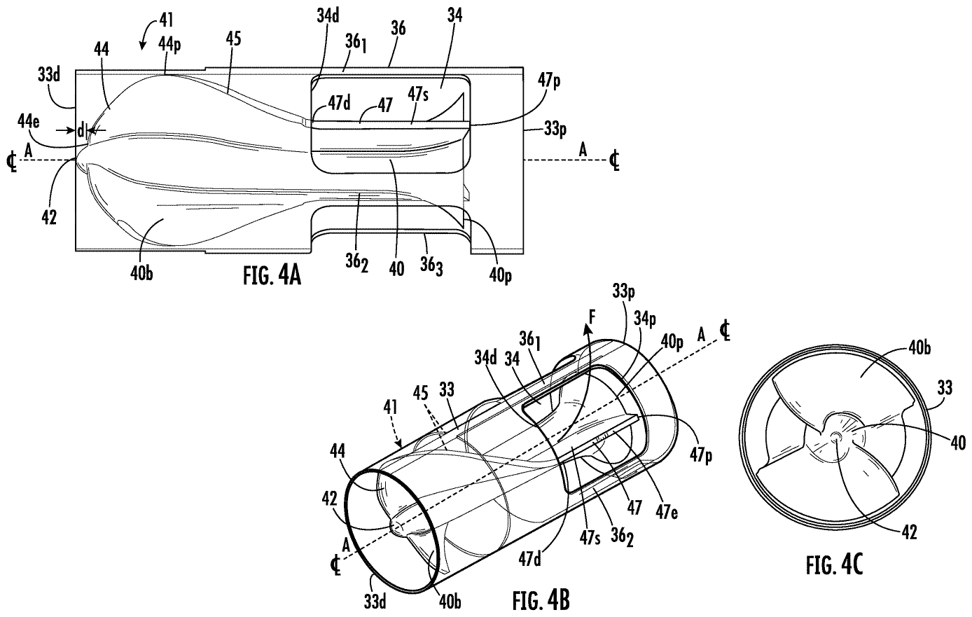

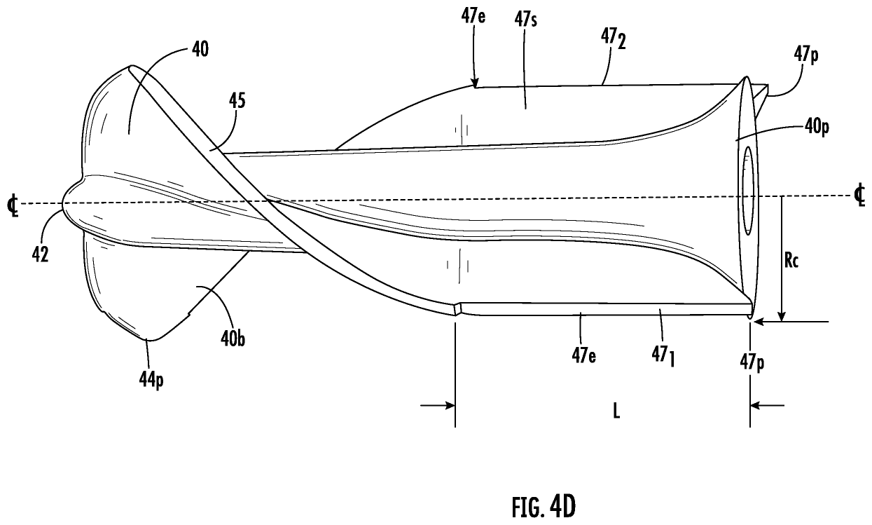

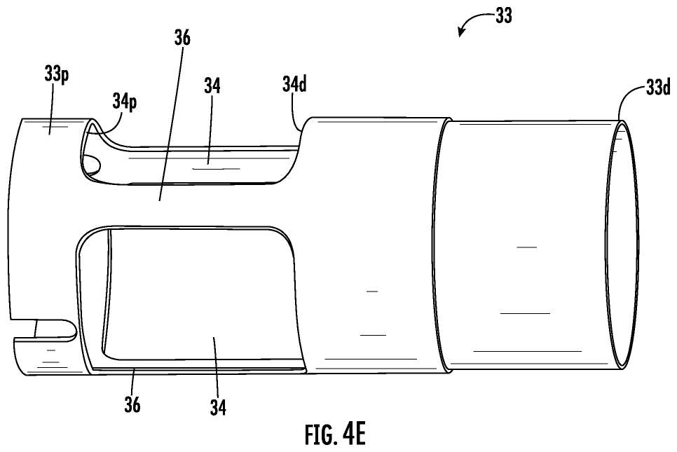

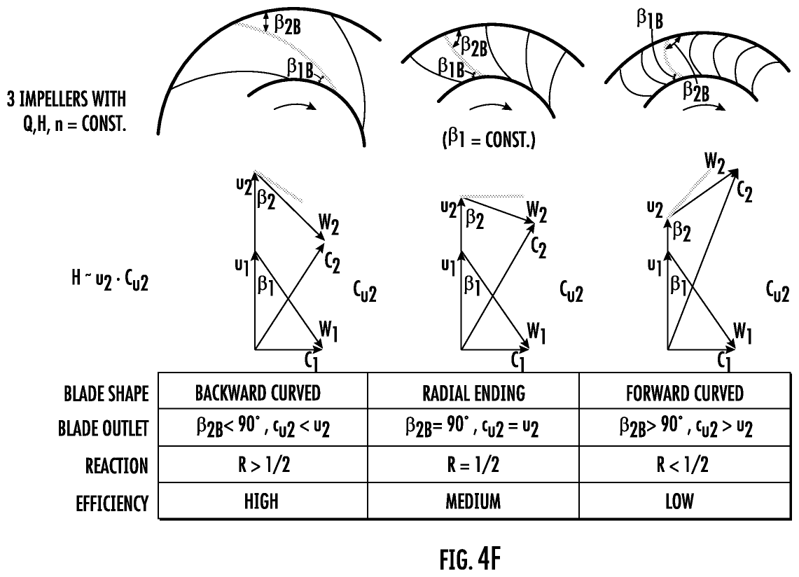

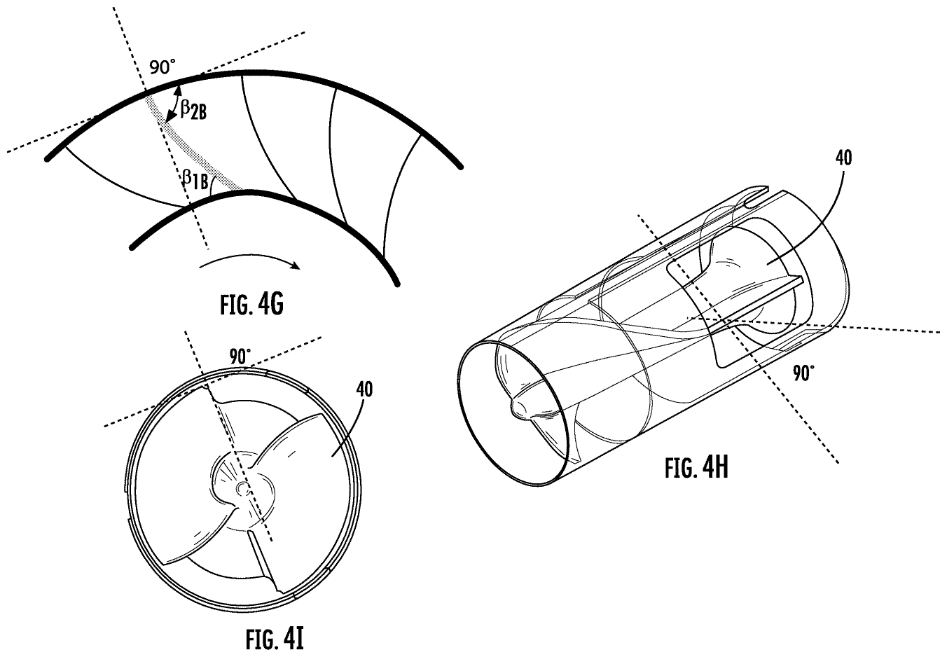

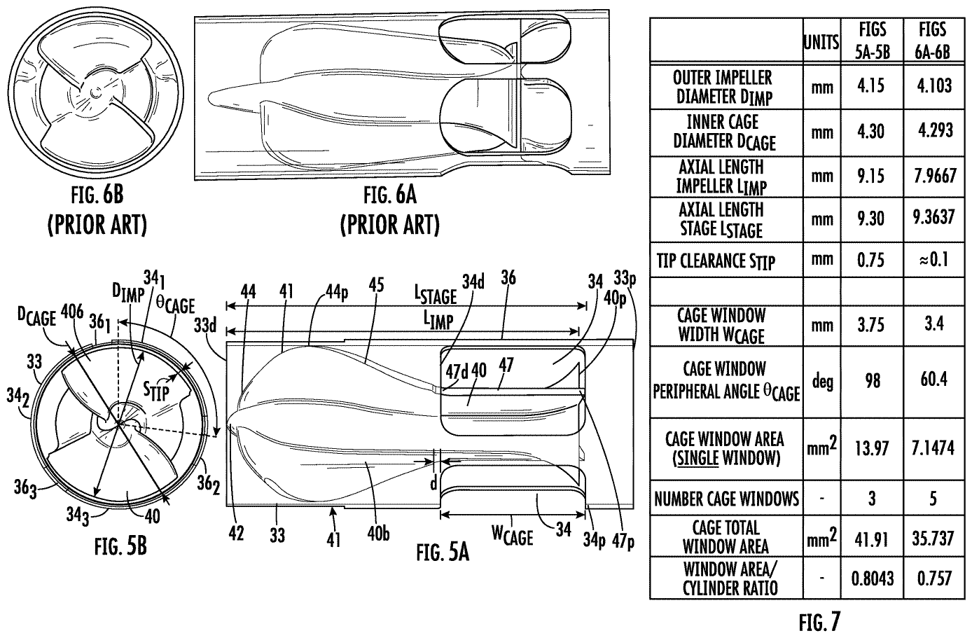

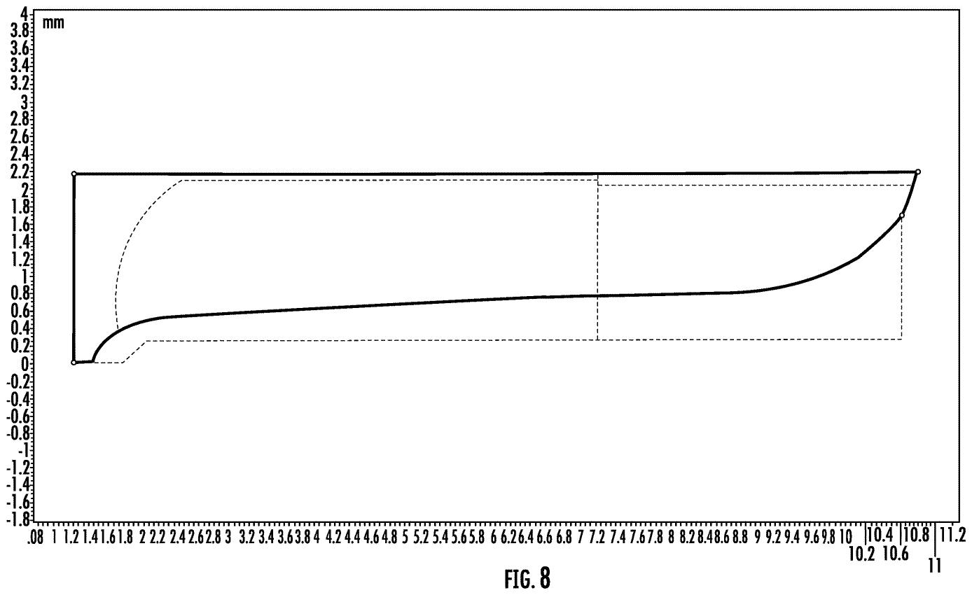

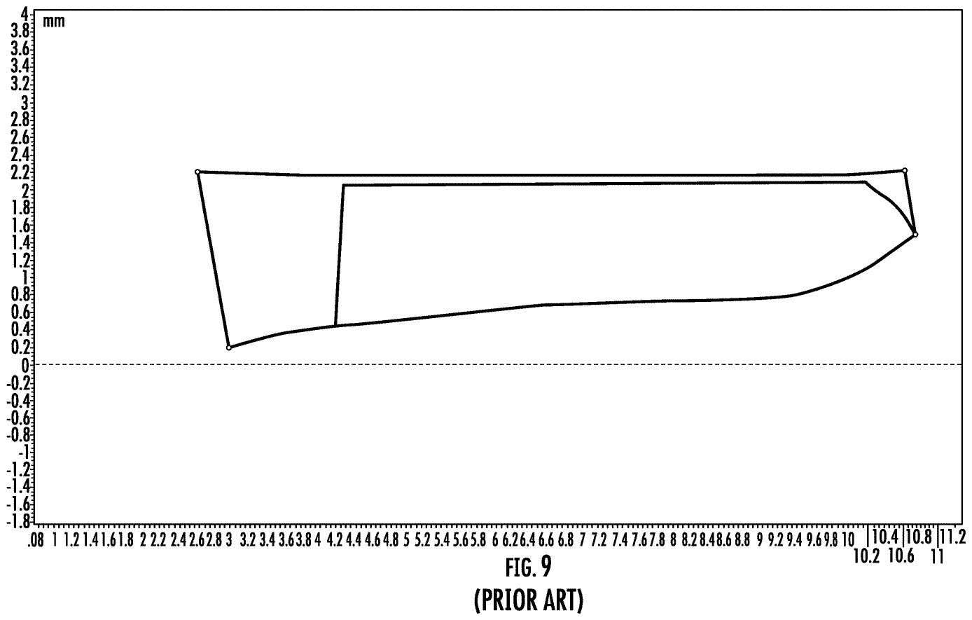

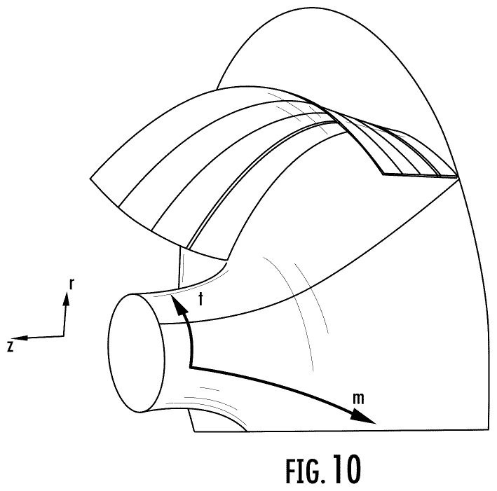

FIGS. 4A-4E show examples of an impeller 40 and impeller cage 33 according to embodiments of the present invention.

FIG. 7 is a table with example geometrical and dimensional features of the devices shown in FIGS. 4A-4C (column adjacent and left of the rightmost column) and the prior art devices shown in FIGS. 3A-3C (rightmost column). As known by those of skill in the art, small changes in dimensions and/or configurations of the impeller and impeller cage and features thereof can have a significant impact on flow rate, hemolysis and/or structural integrity of the device.

Referring to FIGS. 4A-4C, 5A and 5B, the impeller 40 sits in the impeller cage 33. The impeller cage 33 has a plurality of circumferentially spaced apart windows (or openings) 34 separated by respective struts 36.

As shown, the impeller 40 has an impeller body 40b with an impeller blade comprising a curvilinear profile with a pair of vanes 47, each vane 47 providing an exit blade segment 47s that can be aligned to fit in a respective window 34 between neighboring struts 36 of the impeller cage 33. The vane 47 with a respective exit blade segment 47s can project radially outward and have a maximum radial extent that is constant defining a radial most end or edge 47e that extends in a straight linear longitudinal direction for at least 30%, at least 40% or at least 50%, of a length of the window (Wcage, FIG. 5A), with a distal end 47d of the exit blade segment 47 adjacent or longitudinally aligned with a distal end 34d of a window 34. A proximal end 47p of the exit blade segment 47 can terminate adjacent to or longitudinally aligned with a proximal end 34p of the window 34. The vane 47 with the exit blade segment 47s can be structurally rigid and non-deformable during normal operation.

Referring to FIG. 5A, the distal end 47d of the straight segment of the exit blade segment 47s can reside a distance “d” that is closely spaced to the distal end 34d of the window 34, typically distal to but within 0.00 mm to about 0.05 mm. The proximal end 47p of the exit blade segment 47s can reside within 0.00 mm to about 0.05 mm to the proximal end 34p of the window 34. Positioning the straight exit blade segment 47s longitudinally aligned with substantially an entire length Wcage of the windows 34 can provide improved outflow/better hydrodynamic efficiency.

Referring to FIG. 4D, each vane 47₁, 47₂, can provide an exit blade segment 47s that has a constant, maximum radial extent Rc, measured from an axially extending centerline C/L A-A of the impeller body 40b, over a length L providing the straight segment with a. The length L can be in a range of 3.7 to 3.8 mm, such as about 3.75 mm, in some embodiments. Rc can be equal to the maximal outer diameter of the impeller 40. Rc can be about 2.15 mm. Rc can be substantially equal (+/−10%) to a maximal radial extent of peak segment 44p.

Each exit blade segment 47s can define a 90 degree exit surface on all sides of the impeller blade and facing the windows 34 directing blood outwardly toward the windows 34 over an entire longitudinal length thereof, Wcage, which can provide better pump performance.

Referring to FIGS. 4F-4I, the exit blade segment 47s can define an exit blade (outlet flow) angle β2 (β2B) that is equal to 90 degrees and which can provide improved pump performance. FIG. 4F provides a summary of outlet angle calculations for Turbomachinery fundamentals for example parameters with different efficiencies as will be understood by those of skill in the art. Thus, for pumps, the impeller outlet blade angle, which can also be called the “exit blade angle”, describes the angle between the blade (vane) at the outer diameter of the impeller 40 and the circumferential (rotational) direction. FIGS. 4G-4I illustrate a preferred exit blade angle of the impeller 40 according to embodiments of the present invention. As shown by the dashed/broken (virtual) lines in FIGS. 4H and 4I, the exit blade angle is 90 degrees as defined by the intersection of the tangential directions (dashed lines) of the outer diameter and the circumferential (rotational) directions.

The impeller 40 can have only two circumferentially spaced apart vanes 47, each with respective straight (non-curved) exit blade segments 47s with a lesser number of vanes 47 than windows 34, e.g., two impeller vanes 47₁, 47₂, and three windows 34₁, 34₂, 34₃. This configuration can avoid pressure fluctuations which may happen if there were the same number of vanes and struts.

The exit blade segments 47s, adjacent to, at or distal to the distal end 34d of the windows 34, can merge into a curvilinear segment 45 that travels radially outward toward a radially outwardly extending peak 44p segment toward the nose 42 of the impeller blade 40b. The struts 36 can be parallel to the straight impeller exit blade segments 47s of the vanes 47 and a longitudinally extending centerline of the window.

The impeller cage 33 can have only three circumferentially spaced apart windows/openings 34, each with an open window area in a range of 10-14 mm², such as about 13.97 mm² which, based on simulated operational models, can provide lower pressure loss relative to impeller cages with five smaller windows/openings shown in the prior art device of FIGS. 3A-3C, which are about half the size of the larger windows contemplated by embodiments of the invention. The impeller cage 33 can have only three circumferentially spaced apart struts 36₁, 36₂, 36₃ (FIG. 5B). Finite element analyses computations have confirmed that the impeller cage 33 with the three windows 34₁, 34₂, 34₃ and the three struts 36₁, 36₂, 36₃ have sufficient structural integrity for withstanding typical cardiac pulsatile forces and bending moments when placing along a tortuous path to the desired placement in the body.

The three windows 34₁, 34₂, 34₃ can have a total cumulative window area in a range of about 40 mm to about 43 mm, such as about 41.91 mm.

The impeller cage 33 can have a cylinder shape and define an area ratio, WA/CA corresponding to the ratio of the window area/cylinder area of about 0.8043. The window area is defined by a cumulative surface area of the windows 34 and the area of the cylinder is defined by the area of the cylinder portion of the cage 33 surrounding the windows 34 (at the Wcage region of the cage 33, FIG. 5A).

The windows 34 can have an axial length in a range of 3.6 mm-3.8 mm, preferably about 3.75 mm, and a circumferential extent in a range of about 80-98 degrees, preferably in range of about 90-98 degrees, such as about 98 degrees.

The impeller cage 33 can have a proximal end 33p and an axially opposing distal end 33d. The impeller cage 33 can define an axial pump stage length Lstage that extends from the distal end 33d to the proximal end 34p of the window 34 which can be about 9.30 mm. The impeller 40 can have an axial length Limp that is in a range of 9 mm to about 9.15 mm, preferably about 9.15 mm. The impeller length is substantially longer, typically 1 mm or more longer, than the prior art device of FIGS. 3A-3C and, based on simulated operational models, can provide better pump performance.

The impeller 40 has a (distal) tip 42 that resides inside the impeller cage 33 and may reside closely spaced apart from a distal end 33d of the cage 33, inside the cage 33, or flush with the distal end 33d of the impeller cage 33. The tip 42 can reside within a range of 0.07 mm to 0.1 mm, typically about 0.075 mm, from the distal end 33d of the impeller cage 33.

Referring to FIGS. 4A, 5A, the impeller 40, viewed from a side, can have an impeller blade 40b with a curvilinear blade profile 41 extending from the tip 42 in a longitudinal direction to a proximal end 40p of the impeller 40. The curvilinear blade profile 41 has a first curvilinear segment 44 that extends to a radially outwardly projecting peak 44p that merges into a second curvilinear segment 45 that then extends radially inward to merge into the exit blade segment 47. The first curvilinear segment 44 tapers radially outward from an end 44e location, that is close to the tip 42, to the peak 44p. The end location 44e can reside a distance of 0.1 mm to 0.3 mm, typically about 0.285 mm, from the tip 42.

As shown in FIGS. 4A, 5A, the exit blade segment 47 has a constant maximal radial extent from an axially extending centerline A-A of the impeller 40. The exit blade segment 47s can be parallel to the axially extending centerline A-A of the impeller 40 over an entire length of the exit blade segment 47s. The exit blade segment 47 can have a length that is about 50-110% of an axial length Wcage of a respective window 34, more typically 70%-110% of the axial length Wcage.

Referring to FIGS. 5A, 5B, the impeller 40 can have a maximal outer diameter Dimp that is in a range of about 4.1 mm to 4.15 mm, preferably about 4.15 mm. The impeller cage 33 can have an inner diameter “Dcage” that is about 0.15 mm greater than Dimp. In some embodiments, Dcage can be 4.30 mm and Dimp can be 4.15 mm. The outer diameter of the impeller cage 33 can be about 4.545 mm, in some embodiments. The wall thickness (radial direction, front to back dimension) of the impeller cage 33 can be in a range of about 0.0097 inches to about 0.005 inches, in some embodiments). The impeller cage 33 can be metal, such as 304 stainless steel or 316L stainless steel. The blade clearance S_{Tip} at the maximal outer diameter of the impeller (associated with the peak 44p) can be about 0.75 mm.

The impeller 40 can have an axial length Lᵢₘₚ that is in a range of about 8.5 mm to about 9.2 mm, typically about 9.15 mm.

The impeller 40 can have an axial stage length that is about 9.30 mm, which is about 0.05 mm less than the prior art device shown in FIG. 6A.

The impeller 40 can have a large wrap angle that is in a range of about 120 degrees to about 130 degrees, preferably 130 degrees, which is larger than the 113 degree wrap angle of the prior art impeller shown in FIGS. 3A-3C.

Referring to FIG. 10, the impeller 40 can have longer blades relative to the prior art shown in FIGS. 3A-3C, which can increase meridional flow surfaces by about 45 percent. The spatially curved meridional flow surfaces can be mapped to a plane by coordinate transformation. The coordinate system has an angle in the circumferential direction “t” as the abscissa and the dimensionless meridional extension “m” as the ordinate. Both quantities are created by the reference of absolute distances in the meridional (M) and tangential (T) to the local radius “r”:

$$dm = dM / r ;$$

$$dt = dT / r ; and$$

$$tan β = dm / dt ,$$

where “m” is the meridional coordinate, “t” is the tangential coordinate, and m/max is the meridional coordinate related to meridional coordinate at trailing edge.

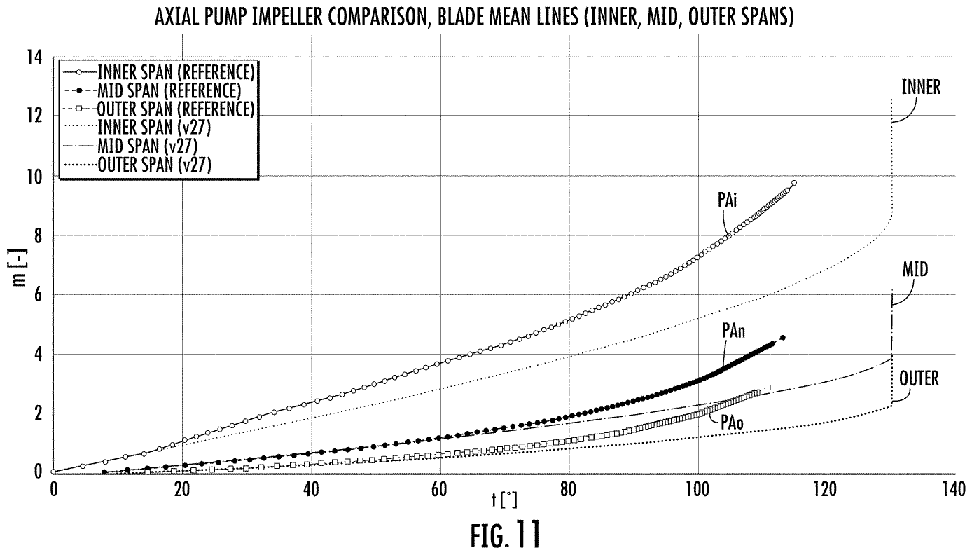

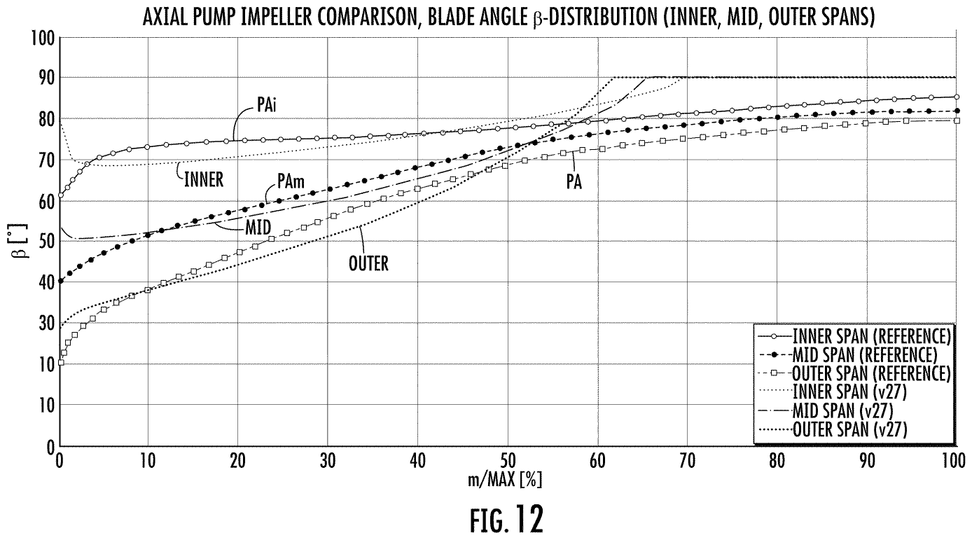

Turning now to FIGS. 11 and 12, graphs of axial pump comparisons of the devices shown in FIGS. 3A and 4A are provided. The lines marked as “PA” and the solid lines with the solid circle markings correspond to the reference/prior art device shown in FIG. 3A. The lines with the broken dashes correspond to the device shown in FIG. 4A.

FIG. 11 shows a graph of blade mean lines (m, t) for the inner, mid, and outer spans of the impeller blades. FIG. 12 shows the blade angle β-distribution, β [° ] versus m/Max [%] for inner, mid, and outer spans of the impeller blades.

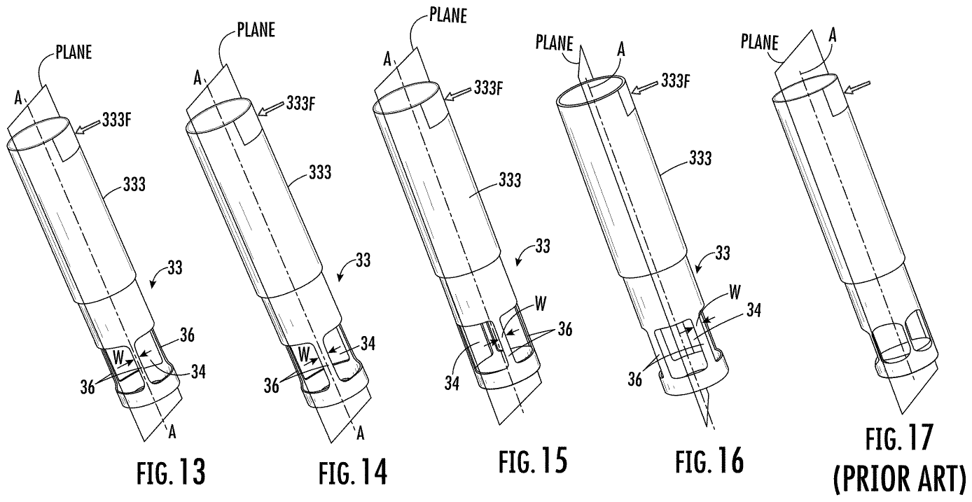

Turning now to FIGS. 13-16, side perspective views of example impeller cages 33 are shown according to some embodiments of the present invention. FIG. 17 is a side perspective view of a prior art impeller cage. FIGS. 13-16 show example struts 36 that are taller and in lesser numbers than the prior art device shown in FIG. 17.

FIG. 13 shows a three strut 36 configuration with each strut 36 having a narrow strut width “w” of about 0.4 mm. FIG. 14 shows a three strut 36 configuration with wider struts than FIG. 13, the struts 36 each having a width “w” of about 0.70 mm.

FIG. 15 shows a four strut 36 configuration with each strut 36 having a narrow strut width “w” of about 0.40 mm. FIG. 16 shows a four strut 36 configuration with wider struts than FIG. 15, the struts 36 each having a width “w” of about 0.70 mm.

A finite element analysis (FEA) was performed on the different configurations of the impeller cages 33 to assess structural integrity under load. The assumptions used for the FEA were that there was a 304 stainless steel (SS) hypo tube cage construction. For analysis of load, a nylon tube was assumed to be bonded to the SS impeller cage and a bending moment is applied to the nylon tube during tortuous insertion through anatomy. The loading is asymmetrical during use and a worst-case deflection direction was assumed for the analysis. Also, fillets were removed, but corner fillets like the prior art device in FIG. 17 were retained. The inside and outside window fillets in FIGS. 13-16 were assumed to have minimal effect on strut strength. The goal of the FEA was to find a yield point of a load applied to the prior art design in FIG. 17 and evaluate the new taller impeller cages with less struts shown in FIGS. 13-16 using that same loading. The loading force 333F was applied to a distal end or the tube 333.

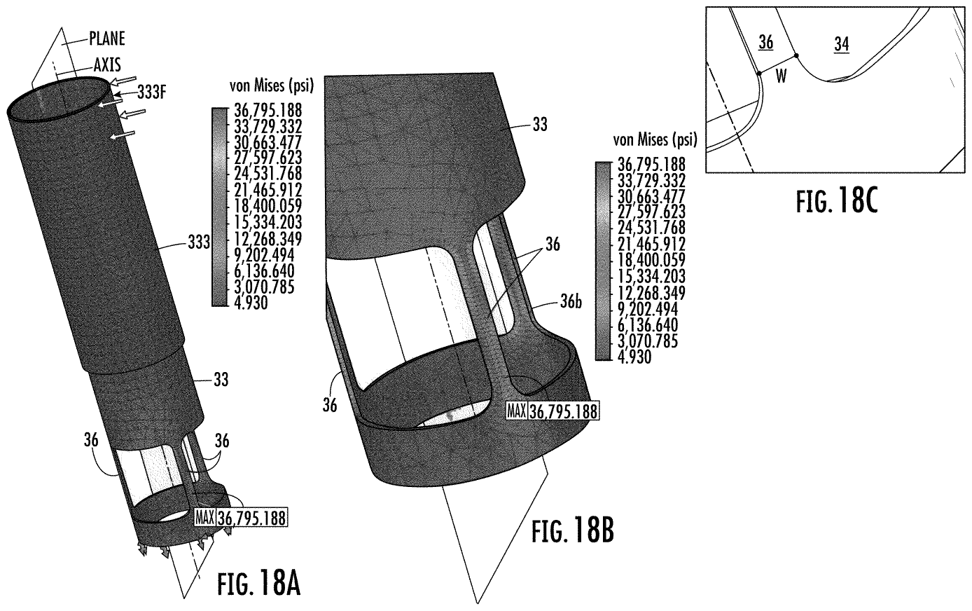

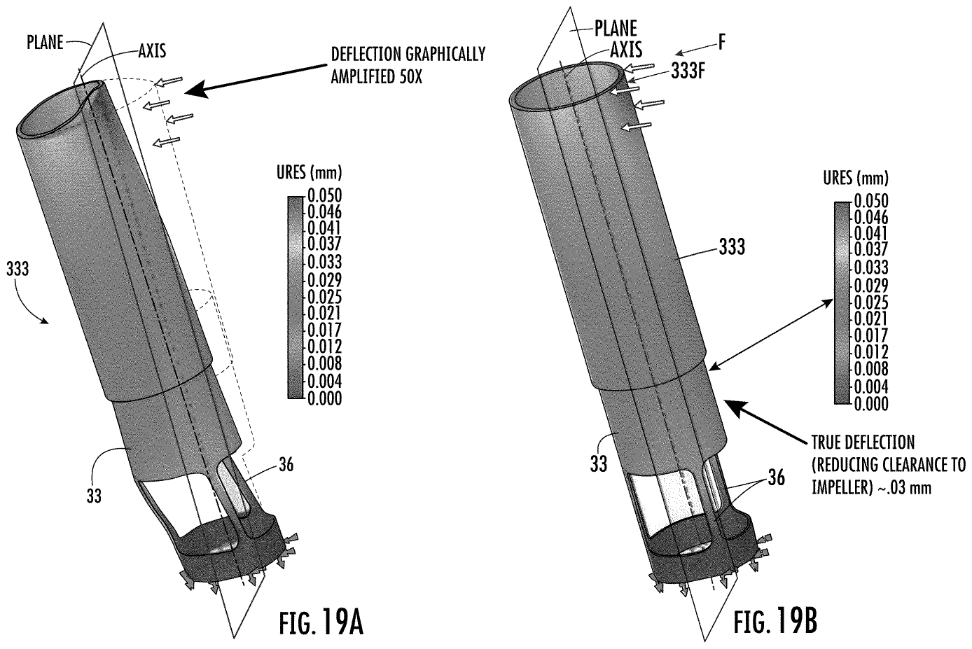

Referring to FIGS. 18A-18C, FEA results of the “narrow” three strut 36 configuration of FIG. 13 is shown with a max yield location identified and with color-coded overlay of yield ranges and legend (von Mises (psi)) with a virtual plane. A load 333F of 0.65 N was applied at the location shown resulting in stresses of 37 Kpsi (yield 32K) at a base 36b of respective struts 36. FIGS. 19A-19B shown the impeller cage 33 and tube 333 graphically deflected at an amplitude of 50×, color-coded to show deflections (URES (mm)). FIG. 19B shows “true” deflection, reducing clearance to the impeller, about 0.03 mm.

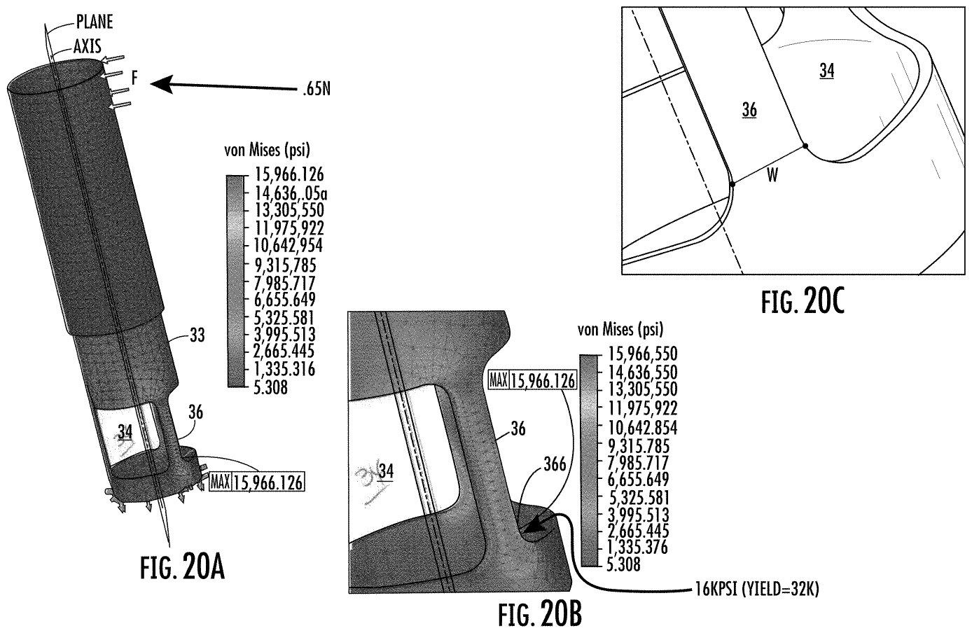

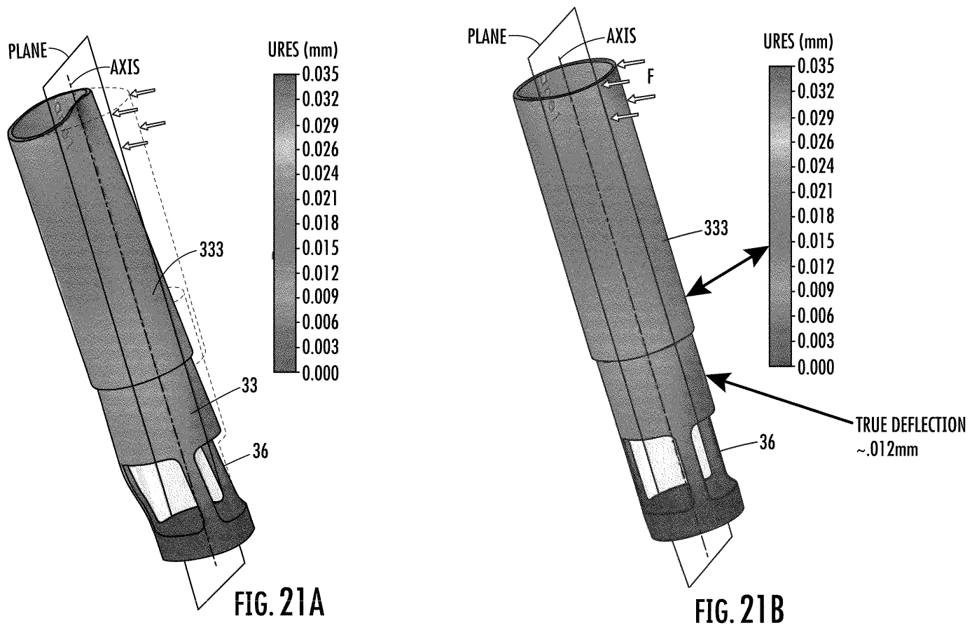

Referring to FIGS. 20A-20C, FEA results of the “wider three strut 36 configuration of FIG. 14 is shown with a max yield location identified and with color-coded overlay of yield ranges and legend (von Mises (psi)) with a virtual plane. A load 333F of 0.65 N was applied at the location shown resulting in stresses of 16 Kpsi (yield 32K) at a base 36b of respective struts 36. FIGS. 21A-21B show the impeller cage 33 and tube 333 graphically deflected at an amplitude of 70×, color-coded to show deflections (URES (mm)). FIG. 21B shows “true” deflection, reducing clearance to the impeller to about 0.012 mm, baseline of about 0.007 mm.

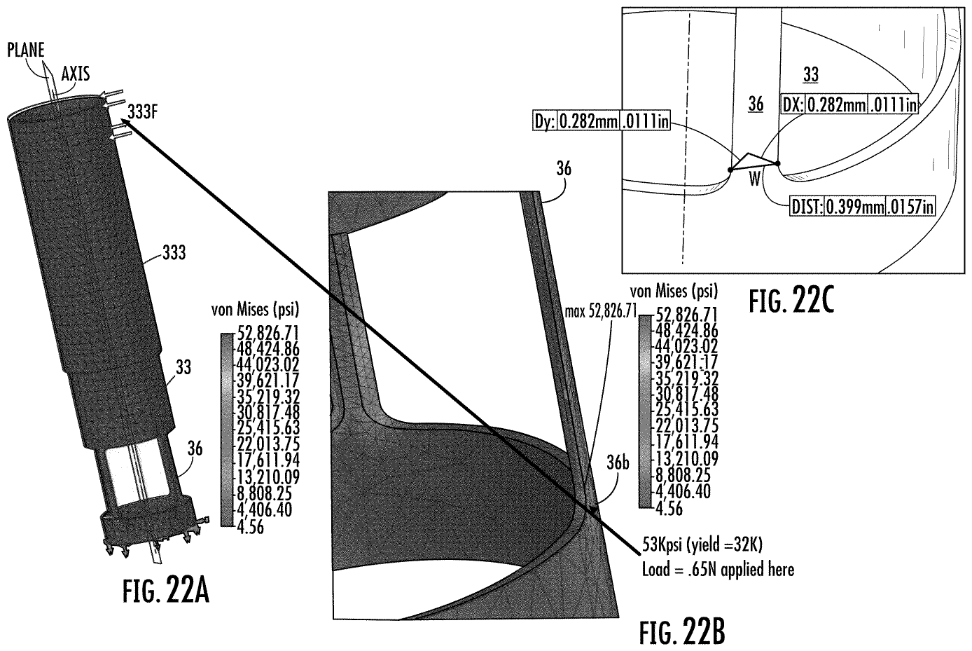

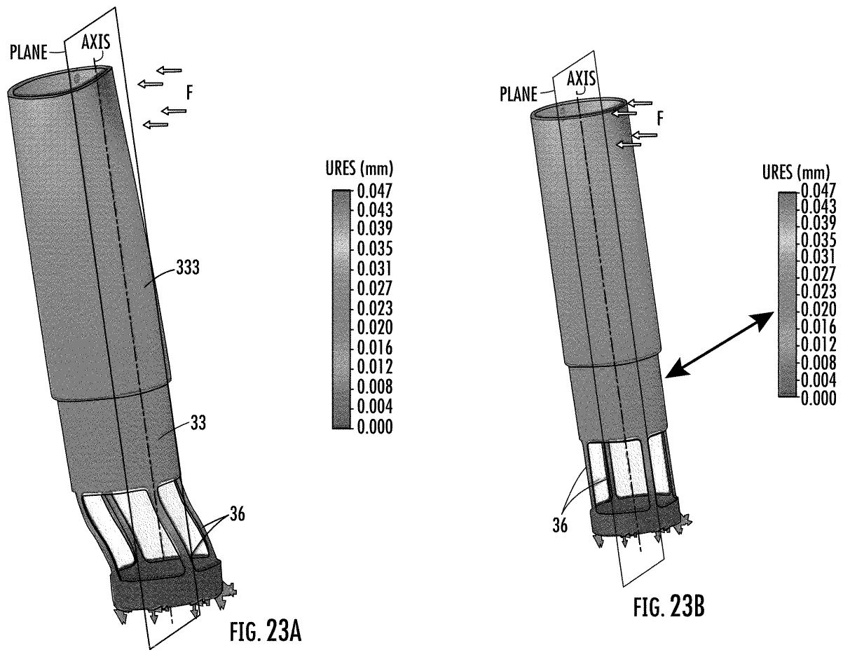

Referring to FIGS. 22A-22C, FEA results of the “narrow” four strut 36 configuration of FIG. 15 is shown with a max yield location identified and with color-coded overlay of yield ranges and legend (von Mises (psi)) with a virtual plane. A load 333F of 0.65 N was applied at the location shown resulting in stresses of 53 Kpsi (yield 32K) at a base 36b of respective struts 36. FIGS. 23A-23B shown the impeller cage 33 and tube 333 graphically deflected at an amplitude of 70×, color-coded to show deflections (URES (mm)). FIG. 23B shows “true” deflection (reducing clearance to the impeller) to about 0.020 mm (baseline of 0.007 mm).

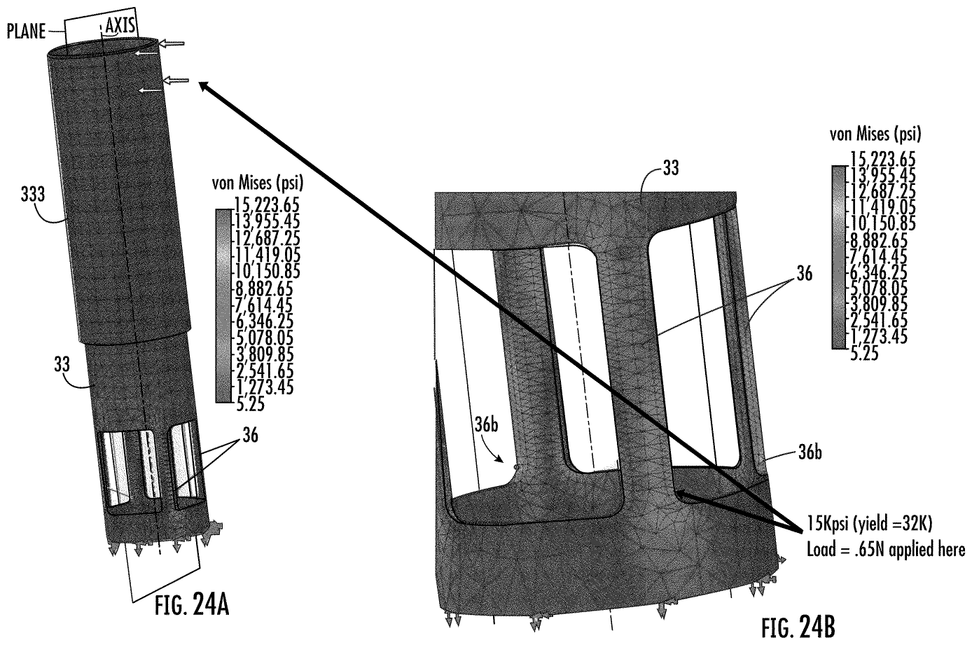

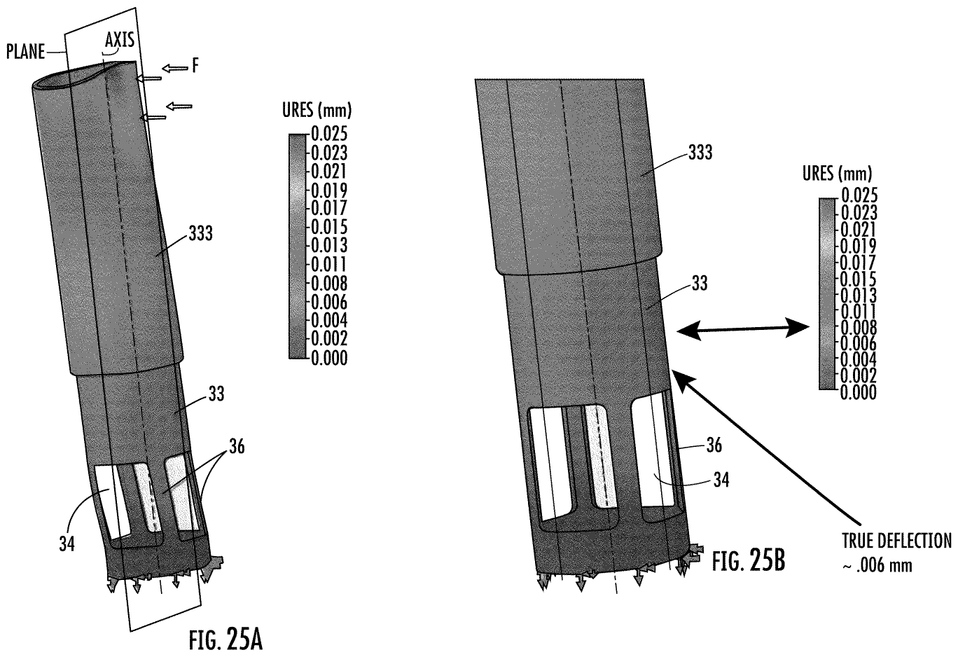

Referring to FIGS. 24A-24C, FEA results of the “wider” four strut 36 configuration of FIG. 16 is shown with a max yield location identified and with color-coded overlay of yield ranges and legend (von Mises (psi)) with a virtual plane. A load 333F of 0.65 N was applied at the location shown resulting in stresses of 15 Kpsi (yield 32K) at a base 36b of respective struts 36. FIGS. 25A-25B shown the impeller cage 33 and tube 333 graphically deflected at an amplitude of 50×, color-coded to show deflections (URES (mm)). FIG. 25B shows “true” deflection (reducing clearance to the impeller), about 0.006 mm (baseline 0.007 mm).

The blood pump 10 can be sized and configured for trans-valvular use, such as for left and/or right ventricular assist procedures. By way of example only, such ventricular assist procedures may be employed in cardiac operations including, but not limited to, coronary bypass graft (CABG), cardiopulmonary bypass (CPB), open chest and closed chest (minimally invasive) surgery, bridge-to-transplant and/or failure-to-wean-from-bypass situations. It is to be readily understood, however, that the intravascular blood pump assembly and methods of the present invention are not to be limited to such applications. Moreover, while illustrated and described largely with reference to left-heart assist applications, it is to be readily understood that the principles of the present invention apply equally with regard to right-heart assist application, which are contemplated as within the scope of the present invention. These and other variations and additional features will be described throughout.

The blood pump 10 can be configured to pump blood through the outlet cage 44 at a rate in a range of about 3.5-7 liters/minute over at least 2 hours, and in other embodiments for several days, such as 6 days or more, of continuous intravascular use. The operational configuration can also continuously providing biocompatible fluid to the in-flow path via at least one in-flow lumen, then to the out-flow path.

The impeller 40 and/or cage 33 can be used with a blood pump 10 configured with an in vivo motor rather than an ex vivo (extracorporeal) motor.

The blood pump 10 may be configured to provide axial or mixed-flow. As used herein, the term “axial flow” is deemed to include flow characteristics which include both an axial and slight radial component.

The blood pump 10 can be configured to provide right and/or left heart support whereby blood is deliberately re-routed through and past the right and/or left ventricle in an effort to reduce the volume of blood to be pumped by the particular ventricle. While “unloading” the ventricles in this fashion is preferred in certain instances, it is to be readily understood that the pump and cannula arrangements described herein may also be employed to “preload” the ventricles. Ventricular preloading may be accomplished by positioning the outflow cage from the pump into a given ventricle such that the pump may be employed to fill or preload the ventricle with blood. This may be particularly useful with the right ventricle. On occasion, the right ventricle is not supplied with sufficient levels of blood from the right atrium such that, upon contraction, the right ventricle delivers an insufficient quantity of blood to the pulmonary artery. This may result when the right ventricle and/or right atrium are in a stressed or distorted condition during surgery. Preloading overcomes this problem by actively supplying blood into the right ventricle, thereby facilitating the delivery of blood into the pulmonary artery. The same technique can be used to preload the left ventricle and thus facilitate the delivery of blood from the left ventricle into the aorta.

In laboratory tests using water as a substitute for blood and a laboratory testing system set up for flow rates (liters per minute) versus pressure (mmHg) at 50,000 rpm over a range of 40 mmHg to 140 mmHg, an impeller and cage corresponding to that shown in FIG. 5A was evaluated as well as the prior art shown in FIG. 6A. The impeller and cage of the present invention yielded improved flow rates over the prior art shown in FIG. 6A over this entire range. Note that while pumping against a 100 mmHg pressure differential, which is clinically typical, catheter blood pumps of the present invention can deliver about 3.7 liters per minute continuous flow which is believed to be a 23.33 percent increase over the prior art shown in FIG. 6A, which is significant and clinically meaningful, potentially representing the difference between recovery or failure to recover.

Hemolysis is red blood cell destruction. The ability to minimize hemolysis is important. Computational model evaluation of the catheter blood pumps of the present invention indicate that they cause the same or less hemolysis than the prior art device shown in FIGS. 3A-3C, even while delivering more flow for longer continuous time periods (not limited to small peak flow outputs). In animal trials of the catheter blood pump (CBP) using the cage and impeller structures shown in FIG. 5A, the animal's hematocrit (a measure of red blood cell volume) did not decrease during the two-hour trial. In humans, a physician typically uses a CBP for two hours or less.

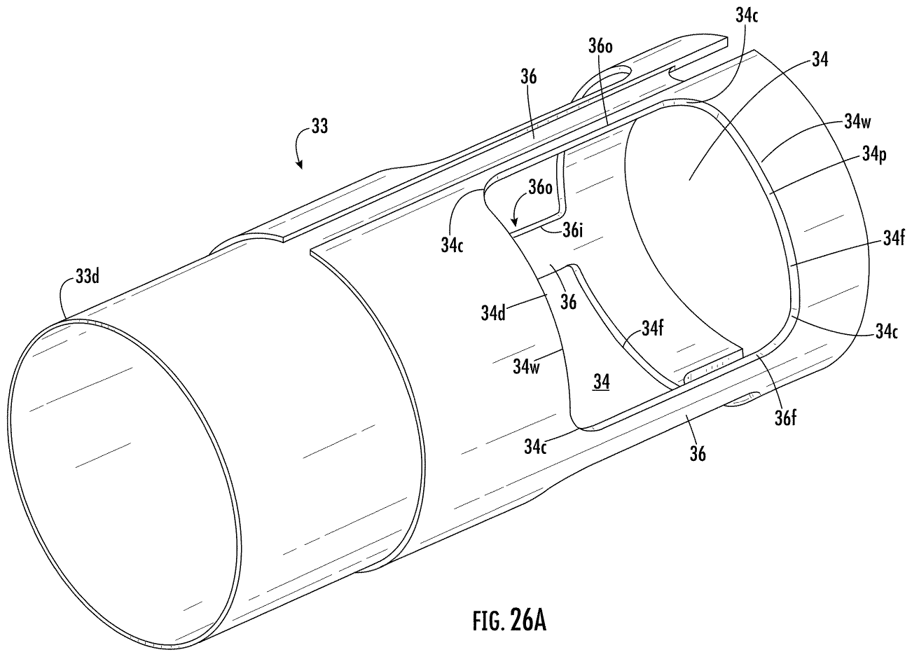

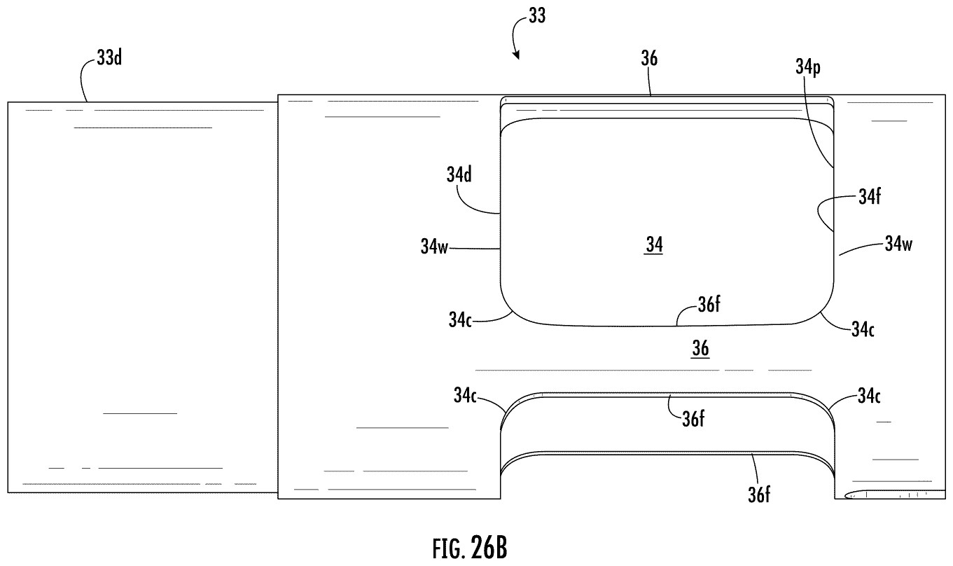

FIGS. 26A and 26B show enlarged views of an example impeller (outlet) cage 33. The windows 34 have a proximal end 34p and a distal end 34d. In this embodiment, pairs of two of the three struts 36 form the long parts of the window perimeter. The relatively large open area of the windows 34 provide a large (unobstructed) exit path for the pumped blood. The struts 36 can have flat faces 36f between an inner edge 36i and an outer edge 36o and the lateral boundary wall 34w of the window 34 can also have flat faces 34f. The windows 34 can have rounded corners 34c that merge the struts 36 with the lateral boundary walls 34w. The windows 34 can have straight lateral segments between the corners 34c (in contrast to the longitudinally arcuate projections of the smaller windows of the prior art housing shown in FIGS. 3A and 6A, for example).

One of the struts 36 can have an axially extending recess or channel that can be used to guide a pressure sensor line to the inflow cage 23 (FIG. 1). The impeller cage 33 can have a stepped configuration so that a distal end portion has a reduced outer diameter relative to the outer diameter at the struts 36, for example. A polymeric and/or plastic tube of the snorkel 21 (FIG. 1) can be adhesively attached or bonded to this reduced outer diameter segment. This snorkel tube 21 can also have an aligned internal or external channel or recess to guide the pressure sensor line to the inflow cage 23.

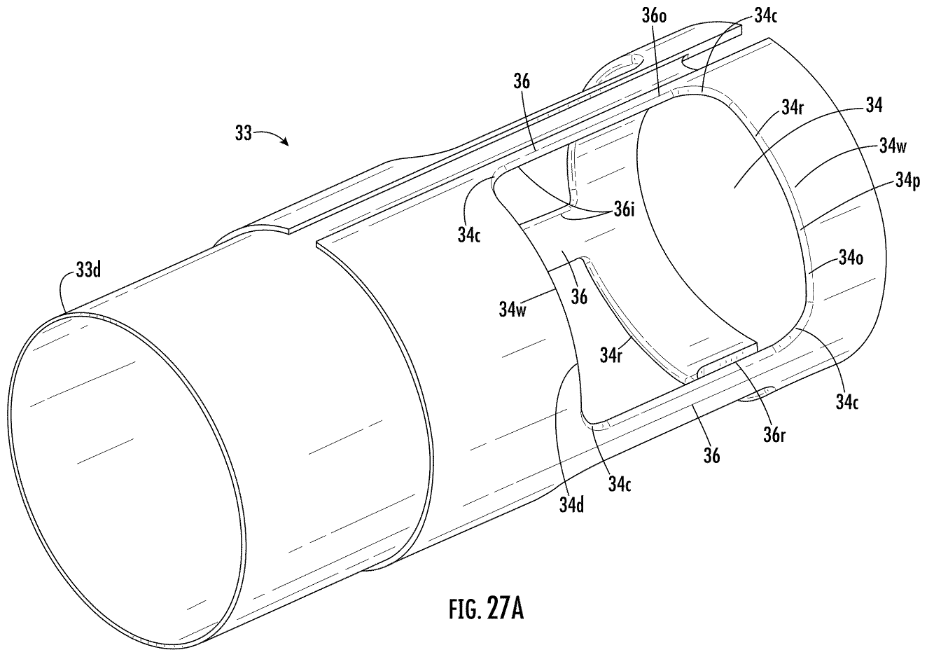

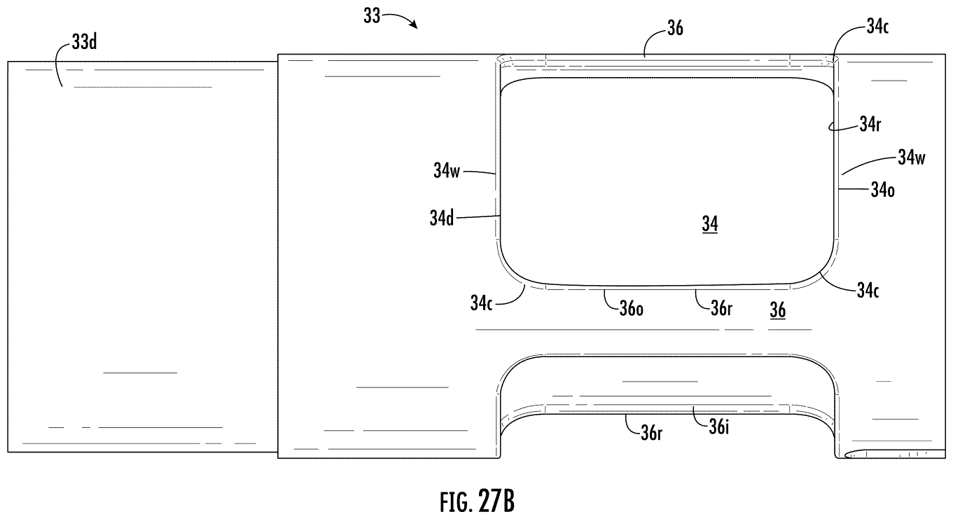

FIGS. 27A and 27B show the impeller (outlet cage) 33, similar to FIGS. 26A and 26B, but instead of flat faces 34f, 36f, rounded faces 36r, 34r are shown. Rounding sharp edges to avoid sharp corners or edges is a well-known manufacturing/design option for devices as will be well known to those of skill in the art.

Notably, in computational model evaluations of the struts 36 in FIGS. 26A, 26B and 27A, 27B, the evaluations showed di minimis differences in hemolysis. This may be because the windows 34 are relatively large and the shape and position of the exit blade 47 (FIG. 6A) is such that the struts 36 do not significantly interact with the blood flow from the exit blade 47 (FIG. 6A) whether the struts 36 have flat faces 36f (FIGS. 26A, 26B) or rounded faces 36r (FIGS. 27A, 27B).

In the drawings and specification, there have been disclosed embodiments of the invention and, although specific terms are employed, they are used in a generic and descriptive sense only and not for purposes of limitation, the scope of the invention being set forth in the following claims.

Thus, the foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention.

Accordingly, all such modifications are intended to be included within the scope of this invention as defined in the claims. In the claims, means-plus-function clauses, where used, are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

1. An impeller for a blood pump, comprising:
an impeller body with a curvilinear profile that extends from a distal nose segment to a pair of vanes positioned proximally of the distal nose segment, wherein each vane provides an exit blade segment that has a constant, maximum radial extent measured from an axially extending centerline of the impeller body over a length of the exit blade segment.

2. The impeller of claim 1, wherein the length is in a range of about 3.7 mm to about 3.8 mm.

3. The impeller of claim 1, wherein the exit blade segment is perpendicular to the axially extending centerline on all spans.

4. The impeller of claim 1, wherein the impeller has an axial length that is about 9.15 mm.

5. The impeller of claim 1, wherein the impeller has a wrap angle of about 130 degrees.

6. The impeller of claim 1, in combination with an impeller cage at least partially surrounding the impeller body, wherein the exit blade segment is longitudinally aligned with cage windows of the impeller cage.

7. The impeller of claim 6, wherein the impeller cage has only three windows, wherein the windows are longitudinally aligned and circumferentially spaced apart, wherein the impeller cage has only three struts, and wherein the three struts are longitudinally aligned and circumferentially spaced apart.

8. The impeller of claim 6, wherein the three windows have a longitudinally extending length and a circumferentially extending width defining a window area, and wherein the window area of each window is in a range of about 10 mm² to about 14 mm².

9. The impeller of claim 6, wherein the exit blade segments have a length that is about 80-110% of a length of the windows, and wherein the exit blade segments define an exit flow angle of 90 degrees.

10. The impeller of claim 8, wherein the length of the exit blade segment is the same as the longitudinally extending length of the three windows.

11. The impeller of claim 6, wherein the cage windows have a circumferential extending peripheral angle that is in a range of 90-100 degrees.

12. The impeller of claim 11, wherein the circumferentially extending peripheral angle is 98 degrees.

13. The impeller of claim 1, wherein the impeller body has an overall length in a range of about 8.5 mm to about 9.5 mm.

14. (canceled)

15. The impeller of claim 1, wherein the impeller tapers from the nose segment proximally outward to a respective peak segment of each of the vanes, wherein the vanes then taper radially inward in a proximal direction to merge into the corresponding exit blades.

16. (canceled)

17. The impeller of claim 11, further comprising an impeller cage surrounding the impeller, wherein a clearance distance between an inner surface of the impeller cage and the peak segments is about 0.75 mm.

18. (canceled)

19. The impeller of claim 6, wherein the impeller cage comprises a window area/cylinder area ratio of 0.8043.

20. The impeller of claim 6, wherein the exit blade segments terminate adjacent a proximal end of the windows.

21. An impeller assembly for a catheter blood pump, comprising:
an impeller comprising a curvilinear profile that extends from a distal nose segment to a pair of vanes extending proximal of the distal nose segment, wherein each vane provides an exit blade segment that has a constant, maximum radial extent measured from an axially extending centerline of the impeller body over a length of the exit blade segment; and
an impeller cage at least partially surrounding the impeller, wherein the exit blade segment is longitudinally aligned with cage windows of the impeller cage.

22. The impeller assembly of claim 21, wherein the impeller cage has only three windows that are longitudinally aligned and circumferentially spaced apart separated by three struts that are longitudinally aligned and circumferentially spaced apart.

23. The impeller assembly of claim 21, wherein the three windows each have a longitudinally extending length and a circumferentially extending width defining a respective window area, and wherein the window area of each window is in a range of 10 mm² to 14 mm².

24. The impeller assembly of claim 21, wherein the exit blade segments have a length that is about 80-110% of a length of the windows, and wherein the exit blade segments define an exit flow angle of 90 degrees.

25. The impeller assembly of claim 21, wherein the length of the exit blade segment is the same as a length of the cage windows.

26. The impeller assembly of claim 21, wherein the cage windows have a circumferential peripheral angle that is in a range of about 90-100 degrees.

27. The impeller assembly of claim 26, wherein the circumferential peripheral angle is 98 degrees.

28. The impeller assembly of claim 21, wherein the impeller has an overall length in a range of about 8.5 mm to about 9.5 mm.

29. (canceled)

30. The impeller assembly of claim 21, wherein the impeller tapers out from the nose segment to first and second peak segments, then tapers radially inward in a proximal direction to merge into the exit blade segments, and wherein the exit blade segments extend along 85-110% of a length of the windows of the impeller cage.

31.-33. (canceled)

34. The impeller assembly of claim 21, wherein the impeller cage comprises a window area/cylinder area ratio of 0.8043.

35. The impeller assembly of claim 21, wherein the exit blade segments terminate adjacent a proximal end of the windows, and wherein the windows have top and bottom perimeters that extend straight in a circumferential dimension.

36. An impeller cage for a catheter blood pump, wherein the impeller cage has only three windows that are longitudinally aligned and circumferentially spaced apart and only three struts that are longitudinally aligned and circumferentially spaced apart with pairs of the three struts on opposing sides of each window.

37-42. (canceled)

43. An in vivo impeller for a blood pump, comprising:
an impeller body configured with a straight exit blade angle of 90 degrees over at least 30% of a length of the impeller body.
